(19) United States
(12) Patent Application Publication
(10) Pub. No.: US 2006/0041260 A1
(43) Pub. Date: Feb. 23, 2006

(54) Fixation system with plate having holes with divergent axes and multidirectional fixators for use therethrough

(76) Inventor: Jorge L. Orbay, Miami, FL (US)

Correspondence Address: GORDON & JACOBSON, P.C., 60 LONG RIDGE ROAD, SUITE 407, STAMFORD, CT, 06902, US

(21) Appl. No.: 11/241,563

(22) Filed: Sep. 30, 2005

Related U.S. Application Data
(63) Continuation-in-part of application No. 10/897,912, filed on Jul. 23, 2004.
Application No. 10/897,912 is a division of application No. 10/159,612, filed on May 30, 2002, now Pat. No. 6,767,351.
Application No. 10/159,612 is a continuation-in-part of application No. 09/739,228, filed on Dec. 19, 2000, now Pat. No. 6,399,497.
Application No. 09/739,228 is a continuation-in-part of application No. 09/495,854, filed on Feb. 1, 2000, now Pat. No. 6,358,250.

(51) Int. Cl. A61F 2/30

(52) U.S. Cl. 606/69

(57) ABSTRACT

A fixation system includes a plate intended to be positioned against a bone and which includes a plurality of threaded holes for receiving the pegs. The threaded holes define respective axes at least two of which are oblique relative to each other. When the pegs are inserted axially through their respective holes, the pegs are relatively divergent from each other. The pegs are also angularly adjustable relative to the hole axes and can be independently fixed in selectable orientations; i.e., the pegs are multidirectional, so as to provide a surgeon selected angular adjustment relative to the hole axes.

### RELATED APPLICATIONS

This application is a continuation-in-part of U.S. Ser. No. 10/897,912, filed Jul. 23, 2004, which is a divisional of U.S. Ser. No. 10/159,612, filed May 30, 2002 and now issued as U.S. Pat. No. 6,767,351, which is a continuation-in-part of U.S. Ser. No. 09/739,228, filed Dec. 12, 2002 and now issued as U.S. Pat. No. 6,440,135, which is a continuation-in-part of U.S. Ser. No. 09/495,854, filed Feb. 1, 2000 and now issued as U.S. Pat. No. 6,358,250.

### BACKGROUND OF THE INVENTION

1. Field of the Invention

This invention relates broadly to surgical devices. More particularly, this invention relates to a bone fixation system having multidirectional bone fragment support pegs.

2. State of the Art

Referring to FIG. 1, a Colles' fracture is a fracture resulting from compressive forces being placed on the distal radius 10, and which causes backward displacement of the distal fragment 12 and radial deviation of the hand at the wrist 14. Often, a Colles' fracture will result in multiple bone fragments 16, 18, 20 which are movable and out of alignment relative to each other. If not properly treated, such fractures result in permanent wrist deformity. It is therefore important to align the fracture and fixate the bones relative to each other so that proper healing may occur.

Alignment and fixation are typically performed by one of several methods: casting, external fixation, interosseous wiring, and plating. Casting is non-invasive, but may not be able to maintain alignment of the fracture where many bone fragments exist. Therefore, as an alternative, external fixators may be used. External fixators utilize a method known as ligamentotaxis, which provides distraction forces across the joint and permits the fracture to be aligned based upon the tension placed on the surrounding ligaments. However, while external fixators can maintain the position of the wrist bones, it may nevertheless be difficult in certain fractures to first provide the bones in proper alignment. In addition, external fixators are often not suitable for fractures resulting in multiple bone fragments. Interosseous wiring is an invasive procedure whereby screws are positioned into the various fragments and the screws are then wired together as bracing. This is a difficult and time consuming procedure. Moreover, unless the bracing is quite complex, the fracture may not be properly stabilized. Plating utilizes a stabilizing metal plate typically against the dorsal side of the bones, and a set of parallel pins extending from the plate into the holes drilled in the bone fragments to provide stabilized fixation of the fragments. However, the currently available plate systems fail to provide desirable alignment and stabilization.

### SUMMARY OF THE INVENTION

It is therefore an object of the invention to provide an improved fixation and alignment system for a Colles' fracture and other fractures.

It is another object of the invention to provide a fixation system which desirably aligns and stabilizes multiple bone fragments in a fracture to permit proper healing.

It is also an object of the invention to provide a fixation system which is highly adjustable to provide a customizable framework for bone fragment stabilization.

In accord with these objects, which will be discussed in detail below, a fracture fixation system is provided which generally includes a plate intended to be positioned against a non-fragmented proximal portion of a fractured bone, a plurality of bone screws for securing the plate along the non-fragmented portion of the bone, and a plurality of bone pegs (or ‘locking screws’) coupled to the plate and extending therefrom into bone fragments adjacent the non-fragment portion.

According to a preferred embodiment of the invention, the plate is generally a T-shaped volar plate defining an elongate body, a head portion angled relative to the body, a first side which is intended to contact the bone, and a second side opposite the first side. The body portion includes a plurality of countersunk screw holes for the extension of the bone screws therethrough. The head portion includes a plurality of threaded peg holes for receiving the pegs therethrough. The threaded holes define respective axes and, according to a preferred aspect of the invention, the respective angles between at least two of the axes are oblique in at least one dimension and preferably two dimensions. As such, in accord with the preferred embodiment, given such orientation of the axes, when the pegs are inserted axially through their respective holes, the pegs are relatively divergent from each other. Moreover, the pegs are angularly adjustable relative to the hole axes and can be independently fixed in selectable orientations; i.e., the pegs are multidirectional, so as to provide a surgeon selected angular adjustment relative to the hole axes.

To stabilize a Colles' fracture, the volar plate is positioned with its first side against the volar side of the radius and bone screws are inserted through the bone screw holes into the radius to secure the volar plate to the radius. The bone fragments are then aligned and, through the peg holes, holes are drilled into and between the bone fragment at angles chosen by the surgeon. The pegs are then inserted into the peg holes and into the drilled holes, and a set screw (or screw cap) is inserted over each peg to lock the peg in the volar plate at the chosen orientation. The volar fixation system thereby stabilizes and secures the bone fragments in their proper orientation.

Given the already divergent axes of the peg holes, which are preselected to approximate the best approach for providing subchondral bone support, a much greater range of angular selection suitable for accommodating the fracture is possible than with holes which are all parallel to each other.

The various adjustably directable pegs can also be used in conjunction with fracture fixation plates adapted for fractures of other bones, e.g., the proximal and distal humerus, the proximal and distal ulna, the proximal and distal tibia, and the proximal and distal femur.

Additional objects and advantages of the invention will become apparent to those skilled in the art upon reference to the detailed description taken in conjunction with the provided figures.

### BRIEF DESCRIPTION OF THE DRAWINGS

FIG. 1 is an illustration of an extremity subject to a Colles' fracture;

FIG. 2 is a top volar view of a right hand volar fixation system according to a first embodiment of the invention;

FIG. 3 is a side view of a volar plate according to the first embodiment of the volar fixation system of the invention;

FIG. 4 is a section view of the head portion of the volar fixation system according to the invention;

FIG. 5 is a proximal perspective view of a bone peg according to an embodiment of the invention;

FIGS. 6 and 7 are proximal and distal perspective views, respectively, of a set screw according to the a first embodiment of the invention;

FIG. 8 is a broken section view of a first embodiment of a directable peg assembly for a fracture fixation system according to the invention;

FIG. 9 is a broken perspective view of a peg and set screw according to the first embodiment of the directable peg assembly of the invention;

FIG. 10 is an illustration of the first embodiment of the volar fixation system provided in situ aligning and stabilizing a Colles' fracture;

FIG. 11 is a broken section view of a second embodiment of a directable peg assembly for a fracture fixation system according to the invention;

FIG. 12 is a broken perspective view of a peg and set screw according to the second embodiment of the directable peg assembly for a fracture fixation system according to the invention;

FIG. 13 is a broken section view of a third embodiment of a directable peg assembly for a fracture fixation system according to the invention;

FIG. 14 is a broken perspective view of a peg and set screw according to the third embodiment of a directable peg assembly for a fracture fixation system according to the invention;

FIG. 15 is a broken section view of a fourth embodiment of a directable peg assembly for a fracture fixation system according to the invention;

FIG. 16 is a broken perspective view of a peg and set screw according to the fourth embodiment of a directable peg assembly for a fracture fixation system according to the invention;

FIG. 17 is a broken section view of a fifth embodiment of a directable peg assembly for a fracture fixation system according to the invention;

FIG. 18 is a broken perspective view of a peg and set screw according to the fifth embodiment of a directable peg assembly for a fracture fixation system according to the invention;

FIG. 19 is a broken section view of a sixth embodiment of a directable peg assembly for a fracture fixation system according to the invention;

FIG. 20 is a broken perspective view of a peg and set screw according to the sixth embodiment of a directable peg assembly for a fracture fixation system according to the invention;

FIG. 21 is a broken section view of a seventh embodiment of directable peg assembly for a fracture fixation system according to the invention; and

FIG. 22 is a broken perspective view of a peg and set screw according to the seventh embodiment of a directable peg assembly of the invention.

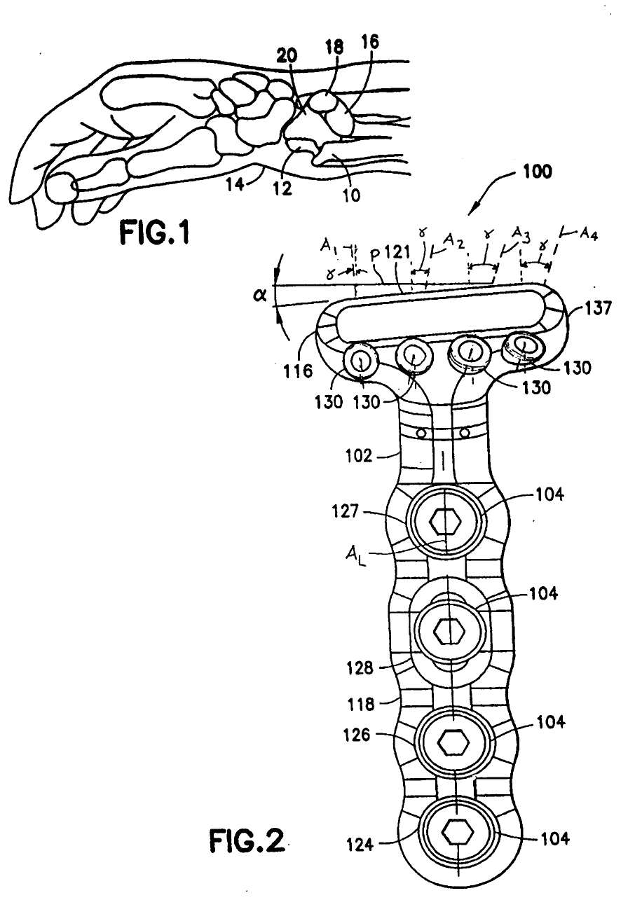

### DETAILED DESCRIPTION OF THE PREFERRED EMBODIMENTS

Turning now to FIGS. 2 through 4, a first embodiment of a fracture fixation system 100 is particularly adapted for aligning and stabilizing multiple bone fragments in a Colles' fracture. The system 100 generally includes a substantially rigid T-shaped plate 102 intended to be positioned against the volar side of the radial bone, a plurality of preferably self-tapping bone screws 104 for securing the plate 102 along a non-fractured portion of the radial bone, and a plurality of bone pegs 108 which extend from the plate 102 and into bone fragments of a Colles' fracture.

The T-shaped plate 102 defines a head portion 116, an elongate body portion 118 angled relative to the head portion, a first side 120 which is intended to contact the bone, and a second side 122 opposite the first side. The first side 120 at the head portion is preferably planar, as is the first side at the body portion. As the head portion and body portion are angled relative to each other, the first side preferably defines two planar portions. The angle Ø between the head portion 116 and the body portion 118 is preferably approximately 23° and bent at a radius of approximately 0.781 inch. The distal edge 121 of the head portion 116 is preferably angled proximally toward the medial side at an angle α, e.g. 5°, relative to a line P, which is perpendicular to a longitudinal axis A_{L} through the body portion. The plate 102 preferably has a thickness of approximately 0.1 inch, and is preferably made from a titanium alloy, such as Ti-6A-4V.

The body portion 118 includes four preferably countersunk screw holes 124, 126, 127, 128 for the extension of the bone screws 104 therethrough. One of the screw holes, 128, is preferably generally elliptical (or oval).

The head portion 116 includes four peg holes (i.e., threaded holes) 130, preferably closely spaced (e.g., within 0.25 inch of each other) and arranged along a line or a curve, for individually receiving the pegs 108 therethrough. The peg holes 130 define respective axes, A_{H}, generally, and A1, A2, A3, A4 respectively, which may be parallel to or angled at respective angles relative to an axis A_{N} normal to the lower surface of the head portion 116 of the plate. According to another preferred aspect of the invention, the respective angles between each of the axes is oblique in two dimensions relative to each other. Thus, angle between any two axes preferably has two components: an angle of rotation γ (FIG. 2) and an angle of inclination β (FIG. 8). Table 1 includes exemplar γ and β data for the hole axes of one embodiment of a volar plate.

TABLE 1
Angular Components of Hole Axes

| Hole Axis | Angle of Rotation (γ) | Angle of Inclination (β) |
|---|---|---|
| A1 | −5° | 0.8° |
| A2 | 1° | 3° |
| A3 | 10° | 7° |
| A4 | 15.5° | 31° |

As such, given the relatively oblique orientation of the axes, when the pegs are inserted axially through their respective holes, the pegs are divergent from each other relative to the lower surface of the head portion.

Further in the accord with the invention, as discussed in more detail below, the pegs are also angularly adjustable relative to the hole axes and can be independently fixed in selectable orientations; i.e., the pegs are multidirectional, so as to provide a surgeon selected angular adjustment relative to the hole axes A_{H}.

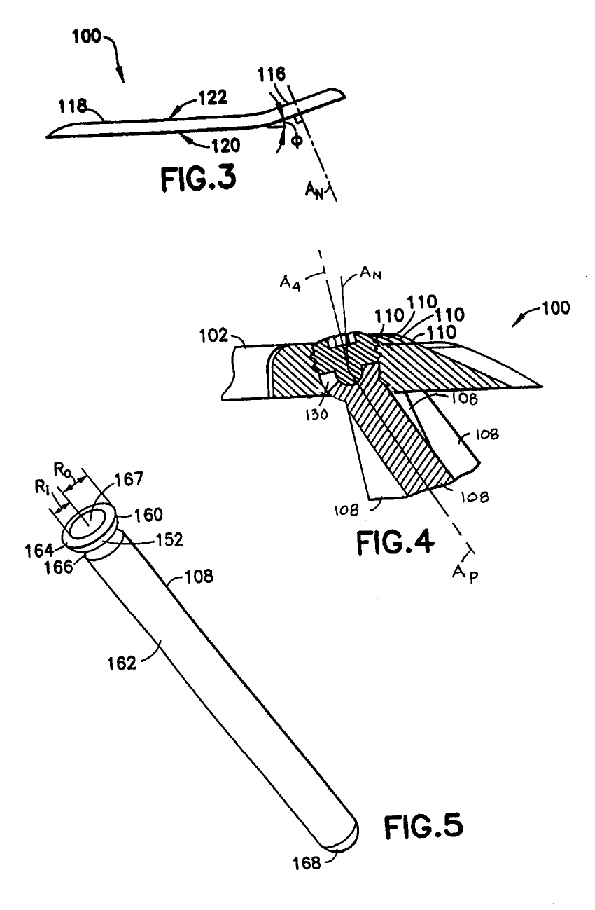

Each peg 108 can be directed through a range of angles within a respective peg hole and fixed at a desired angle within the range. Referring to FIGS. 4 and 8, according to a first embodiment of the invention, each peg hole 130 in the volar plate 102 includes a cylindrical upper bore 140 provided with threads 146 (defining a hole axis A_{H}) and a lower portion 148 having a radius of curvature. The lower end 154 of each peg hole includes a circumferential bevel 156.

Referring to FIGS. 4, 5 and 8, each peg 108 includes a head 160 and a cylindrical shaft 162. The proximal portion 164 of the head 160 includes a cup 167 having an outer radius R_{O} substantially corresponding to the radius of the lower portion 148 of the peg holes 130, and a relatively smaller inner radius Rᵢ of curvature. The head 160 defines preferably approximately 160° of a sphere. The shaft 162 includes a slight taper 166 at the intersection with the head 160, and a rounded distal end 168. According to a preferred manufacture of the first embodiment, the cylindrical shaft 162 of each peg 108 is first provided with a sphere (not shown) or a hemisphere (not shown) at a proximal end. If a sphere is provided, it is cut to a hemisphere. The hemisphere is then hollowed and further reduced to the 160° shape. Finally, the taper 166 is provided at the intersection.

Referring to FIGS. 5 and 8, the surface 150 of the lower portion 148 of the peg hole 130 and/or the outer surface 152 of the head 160 of the peg 108 is preferably roughened, e.g., by electrical, mechanical, or chemical abrasion, or by the application of a coating or material having a high coefficient of friction.

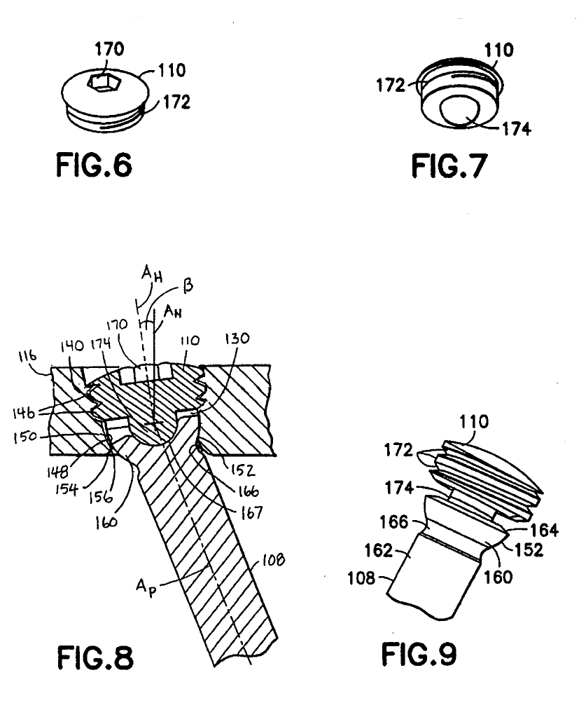

Turning now to FIGS. 6 through 9, each set screw 110 includes a proximal hex socket 170, circumferential threads 172 adapted to engage the threads 146 of the upper bore 140 of the peg hole, and distal substantially hemispherical portion 174 having substantially the same radius of curvature as the inner radius of curvature Rᵢ of the cup 167, and preferably substantially smaller than a radius of the peg holes 130.

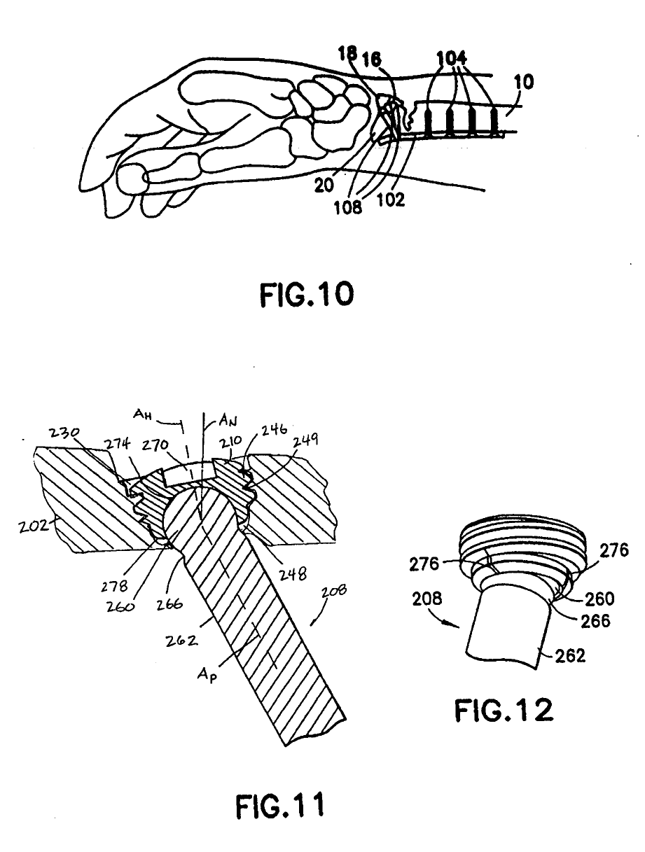

Referring to FIGS. 4 and 10, according to one method to stabilize a Colles' fracture, the plate 102 is positioned on the radius 10 and a hole is drilled through the elliptical screw hole on the volar plate and into the radius 10. Then, a bone screw 104 is inserted through the plate and into the bone and gently secured. The fractured bones 16, 18, 20 are then adjusted under the plate 102 into their desired stabilized positions. Then, through the peg holes 130, the surgeon drills holes into the fracture location for the stabilization pegs 108. The holes may be drilled at any angle within a predefined range, and preferably at any angle within a range of 15° relative to the respective peg hole axis A_{H}. After each hole is drilled, a peg 108 is inserted therein. Referring back to FIG. 8, the bevel 156 at the lower end 154 of the peg hole 130 and the taper 166 on the shaft cooperate to permit the peg to be oriented (along axis A_{P}) with greater freedom relative to the axis A_{H}, if required, as interference between the peg hole and peg shaft is thereby reduced. Once the peg 108 has been appropriately positioned within the peg hole, one of the set screws 110 is threaded into the upper bore 140 of the peg hole 130. The hemispherical portion 174 contacts the head 160 of the peg, seating in the concavity of the cup 167. As the set screw 110 is tightened, the surface 152 of the head of the peg, which may be roughened, is clamped between the set screw 110 and the roughened inner surface 150 of the lower portion of the peg hole 130, thereby securing the peg in the selected orientation. The other pegs are similarly positioned and angularly fixed.

Turning now to FIGS. 11 and 12, a second embodiment of a directable peg assembly for a fracture fixation plate is shown. The plate 202 includes threaded peg holes 230 that are generally larger in diameter than the head 260 of the pegs 208 intended for use therethrough. This permits a hole to be drilled through the peg hole 230 at a relatively greater angle than with respect to holes 130. The lower end 248 of the peg hole 230 is constricted relative to the upper threaded portion 249. The peg 208 includes a spherically-curved head 260, a cylindrical shaft 262, and an optionally constricted neck 266 therebetween. The set screw 210 includes a square opening 270 adapted to receive a square driver, threads 246 about its circumference, and a substantially spherically curved socket 274 adapted to receive the head 260 of the peg 208. As seen in FIG. 12, the lower portion of the set screw 210 includes expansion slots 276 which permit the lower portion of the set screw 210 to temporarily deform to receive the head 260 of the peg 208 (which has a diameter greater than the opening 278 of the spherical socket); i.e., the head 260 can be snapped into the socket 274.

In use, for each peg hole 230 and peg 208, holes are drilled through the peg holes along respective axes A_{P} and into the bone for the pegs stabilize the bone fragments. The head 260 of the peg 208 is snapped into the opening 278 of the socket 274. The shaft 262 of the peg 208 is then inserted into a respective bone hole until the set screw 210 meets the peg hole 230. It is appreciated that the set screw 210 can be rotated (along axis A_{H}) relative to the peg 208, as the socket 274 and spherical head 260 form a ball and socket coupling. As such, the set screw 210 is rotatably secured in the peg hole 230 to secure the peg 208 at the desired angle within the drilled hole.

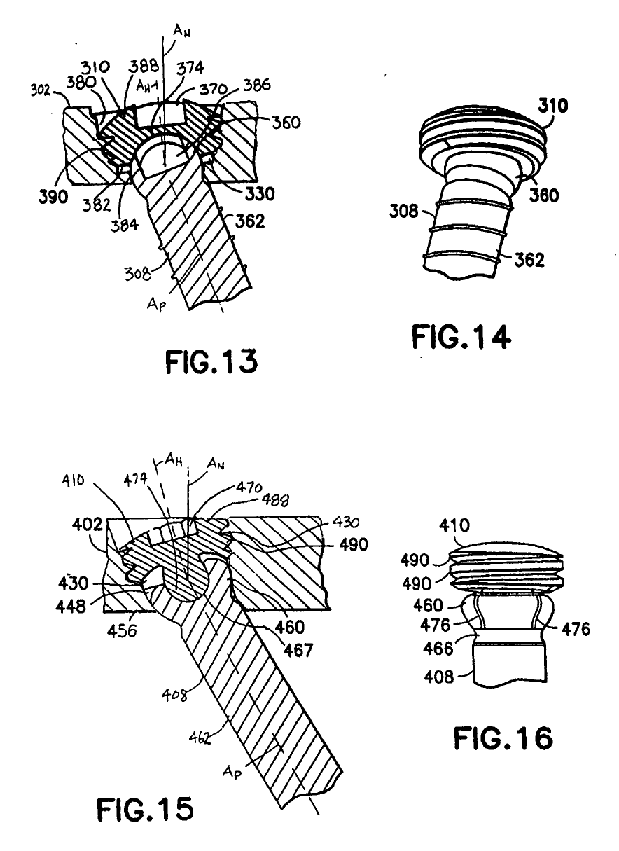

Turning now to FIGS. 13 and 14, a third embodiment of a directable peg assembly for a fracture fixation plate is shown. The plate 302 includes threaded peg holes 330 that preferably each have a stepped diameter, with a relatively large countersink portion 380 adapted to receive the head of a set screw 310, a threaded central portion 382, and a relatively smaller lower portion 384. The peg 308 includes a substantially spherically-curved head 360 having a central square opening 386 adapted to receive a driver, and a threaded cylindrical shaft 362. The set screw 310 includes a head portion 388 having a square opening 370 for also receiving a driver, a threaded portion 390, and a lower spherically-curved socket 374.

In use, for each peg hole 330 and peg 308, a hole is drilled through a respective peg hole and into the bone along axis A_{P} (i.e., at the angle at which it is desired to receive a peg for stabilization of the fracture). The peg 308 is then positioned within the peg hole 330 and rotatably driven into the bone with a driver (not shown). Once the peg 308 is fully seated against the lower portion 384 of the peg hole 330, the set screw 310 is threaded into the central portion 390 of the peg hole in alignment with axis A_{H} and urged against the head 360 of the peg 308 to clamp the peg in position. The head portion 388 of the set screw 310 preferably at least partially enters the countersink portion 380 of the peg hole to provide a lower profile to the assembly.

Turning now to FIGS. 15 and 16, a fourth embodiment of a directable peg assembly for a fracture fixation plate is shown. The plate 402 includes threaded peg holes 430 each with a lower portion 448 having a radius of curvature, and a lower end 454 provided with a preferably circumferential bevel 456. The peg 408 includes a spherically curved head 460 defining a socket 467 extending in excess of 180°, a cylindrical shaft 462, and an optionally tapered neck 466 therebetween. The head 460 about the socket 467 is provided with expansion slots 476. The set screw 410 includes an upper portion 488 having a square opening 470 for a driver, and threads 490, and a lower ball portion 474 adapted in size an curvature to snap into the socket 467.

In use, for each peg hole 430 and peg 408, holes are drilled through the peg hole and into the bone along axes A_{P} (i.e., at the angle at which it is desired to receive a peg for stabilization of the fracture) at the angles at which it is desired to receive pegs for stabilization of fragments of the fracture. The ball portion 474 of the set screw 410 is snapped into the socket 467, with the socket 467 able to expand to accept the ball portion 474 by provision of the expansion slots 476. The shaft 462 of the peg 408 is then inserted into a respective bone hole until the set screw 410 meets the peg hole 430. It is appreciated that the set screw 410 can rotate relative to the peg 408, as the ball portion 474 and socket 467 are rotatably coupled to each other. The set screw 410 is then rotatably secured in the peg hole 430 in alignment with axis A_{H} to secure the peg 408 in the bone.

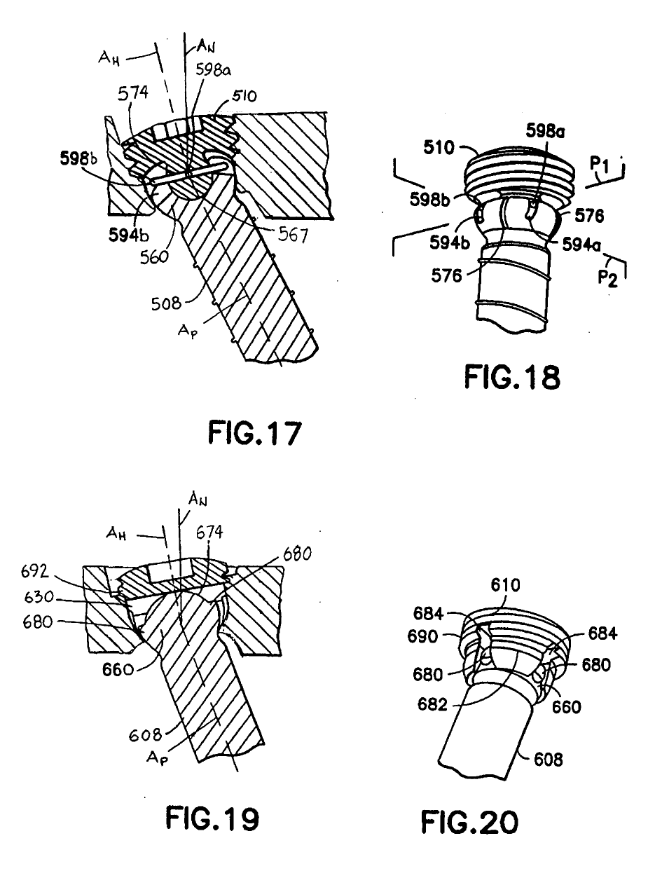

Turning now to FIGS. 17 and 18, a fifth embodiment of a directable peg assembly for a fracture fixation plate, substantially similar to the fourth embodiment, is shown. In the fifth embodiment, the head 560 of the peg 508 includes two sets of pin slots 594a, 594b defining two planes P₁ and P₂ oriented transverse to each other. In addition, the head 560 includes radial expansion slots 576. The ball portion 574 of the set screw 510 includes two pins 598a, 598b extending through a center thereof and oriented transverse to each other. The ball portion 574 is snapped into the socket 567 defined by the head 560 of the peg 508, and pins 598a, 598b are positioned through the pin slots 594a, 594b to rotatably lock the peg and set screw together, yet permit the peg 508 to articulate relative to the set screw 510. The fifth embodiment is suitable for rotatably inserting threaded pegs 508 into a bone hole via rotation of the set screw 510, and may be used in a similar manner to the fourth embodiment.

Turning now to FIGS. 19 and 20, a sixth embodiment of a directable peg assembly for a fracture fixation plate is shown. The assembly includes a peg 608 having a substantially spherically curved head 660 provided with four nubs 680 (two shown) arranged in 90° intervals about the periphery of the head 660. The set screw 610 includes lower walls 682 defining a socket 674 for the head 660. In addition, the walls 682 define slots 684 through which the nubs 680 can move.

In use, the assembly of the peg 608 with its set screw 610 functions substantially similar to a universal joint. The peg 608 is then inserted through a respective peg hole 630 and into a hole drilled into a bone along axis A_{P} until threads 690 on the set screw 610 engage mating threads 692 in the peg hole. The set screw 610 is then rotated to advance the set screw in alignment with axis A_{H}, which causes rotation of the peg 608 within the drilled hole. When the set screw 610 is fully seated in the peg hole 630, the peg 608 is secured in the bone.

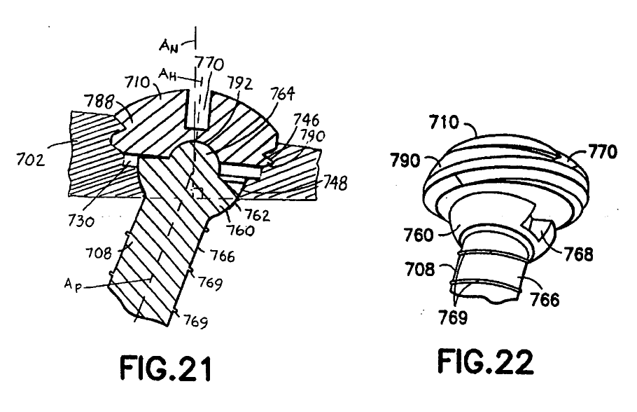

Turning now to FIGS. 21 and 22, a seventh embodiment of a directable peg assembly for a fracture fixation plate is shown. The peg holes 730 in the plate 702 each include a threaded cylindrical upper portion 746 and a spherically-curved lower portion 748 having a smaller hole diameter than the upper portion. Each peg 708 has a head 760 with a lower relatively larger spherically curved portion 762 and an upper relatively smaller spherically curved portion 764, and a shaft 766. The lower curved portion 762 preferably spherically curves through substantially 150°, while the upper curved portion 764 preferably spherically curves through substantially 210°. The peg shaft is optionally provided with threads 769, and when so provided, the lower curved portion 762 of the peg is provided with driver notches 768. The set screw 710 includes an upper portion 788 having a slot 770 or other structure for engagement by a driver, threads 790, and a lower socket 792.

In use, for each peg 708, a hole is drilled through a respective peg hole into the bone along axis A_{P} (i.e., at an orientation desirable for receiving that particular peg for stabilization of the fracture). A peg 708 is then inserted through the peg hole 730 and into the drilled hole until the curved lower surface 762 of the head 760 of the peg seats against the curved lower portion 748 of the peg hole. If the peg has threads 769, a driver (not shown) may be coupled to the peg 708 at the notches 768 to rotationally drive the peg into the drilled hole. The set screw 710 is then threaded and advanced into the peg hole 730 in alignment with axis A_{H} until the socket 792 extends over the upper portion 764 of the head 760 of the peg and presses thereagainst to force the lower portion 762 of the head against spherically-curved lower portion 748 of the peg hole 730 to clamp the peg 708 in position. In the seventh embodiment, the socket 792 of the set screw 710 does not necessarily capture (i.e., extend more than 180° about) any portion of the head 760 of the peg 708. However, the socket 792 may be modified to enable such capture.

There have been described and illustrated herein several embodiments of a volar fixation system, as well as directable peg systems in which the holes for the pegs have relatively oblique axes. In each of preferred embodiments, the head of a peg is clamped between a portion of the fixation plate and a set screw, preferably with the head of the peg and fixation plate thereabout being treated to have, or having as material properties, high friction surfaces. While particular embodiments of the invention have been described, it is not intended that the invention be limited thereto, as it is intended that the invention be as broad in scope as the art will allow and that the specification be read likewise. Thus, while particular materials for the elements of the system have been disclosed, it will be appreciated that other materials may be used as well. In addition, fewer or more peg holes and bone pegs may be used, preferably such that at least two pegs angled in two dimensions relative to each other are provided. Also, while a right-handed volar plate is described with respect to an embodiment of the invention, it will be appreciated that right- or left-handed model, with such alternate models being mirror images of the models described. In addition, while it is disclosed that the pegs may be directed through a range of ±15° relative to axis A_{H}, the peg holes and pegs may be modified to permit a greater range, e.g. up to ±30°, or smaller range, e.g. ±5°, of such angular orientation. Furthermore, while several drivers for applying rotational force to set screws and pegs have been disclosed, it will be appreciated that other rotational engagement means, e.g., a Phillips, slotted, star, multi-pin, or other configuration may be used. Also, the plate and pegs may be provided in different sizes adapted for implant into different size people. Furthermore, while four screw holes are described, it is understood that another number of screw holes may be provided in the plate, and that the screw holes may be located at positions other than shown. In addition, individual aspects from each of the embodiments may be combined with one or more aspects of the other embodiments. Moreover, while some elements have been described with respect to the mathematically defined shapes to which they correspond (e.g., spherical), it is appreciated that such elements need only correspond to such shapes within the tolerances required to permit the elements to adequately function together; i.e., the elements need only be “substantially” spherical in curvature. Furthermore, the concepts provided herein may be applied to fixation systems for other fractures, particularly of other long bones e.g., the humerus, the clavicle, the femur, and the tibia. It will therefore be appreciated by those skilled in the art that yet other modifications could be made to the provided invention without deviating from its spirit and scope as claimed.

1. A fracture fixation system, comprising:
a) a substantially rigid plate having a bone contacting surface and a plurality of holes having an internal threaded, said holes defining at least two axes which are oblique relative to each other;
b) a plurality of pegs each having a head and a shaft, said shaft sized to be received through said threaded holes, wherein said pegs when received within said threaded holes are capable of being in an angular orientation relative to respective axes through said holes; and
c) a plurality of set screws engageable with said internal threads of said threaded holes for applying a force to said heads of said pegs to limit angular movement of said pegs relative to said holes.

2. A fracture fixation system according to claim 1, wherein:
said plate is a sized and shaped for placement at the distal volar radius.

3. A fracture fixation system according to claim 1, wherein:
said threaded holes includes at least three threaded holes in substantially linear arrangement.

4. A fracture fixation system according to claim 3, wherein:
said threaded holes are arranged in a generally medial to lateral direction.

5. A fracture fixation system according to claim 1, wherein:
said at least two axes are divergent from each other in at least one dimension.

6. A fracture fixation system according to claim 1, wherein:
said at least two axes are divergent from each other in at least two dimensions.

7. A fracture fixation system according to claim 1, wherein:
said at least two axes are oblique relative to each other in at least two dimensions.

8. A fracture fixation system according to claim 7, wherein:
said at least two axes are relatively angled in rotation and inclination.

9. A fracture fixation system according to claim 1, wherein:
each of said axes is obliquely angled relative to the other axes.

10. A fracture fixation system according to claim 9, wherein:
each of axes is angled relative to the other in rotation and inclination.

11. A fracture fixation system according to claim 1, wherein:
said plate including a body portion and head portion angled relative to said body portion, said body portion including at least one screw hole.

12. A fracture fixation system according to claim 1, wherein:
said threaded holes having an upper portion and a lower portion, said upper portion having said internal thread, and said lower portion having a radius of curvature, and
said head of said pegs having a lower surface with substantially a same radius of curvature as said lower portion of said threaded holes.

13. A fracture fixation system according to claim 1, wherein:
when said pegs are fixed at their respective angles, said pegs are adapted to provide a framework for supporting fractured bone fragments, said pegs of said framework defining a plurality of non-parallel axes.

14. A fracture fixation system according to claim 1, wherein:
said lower portion of each said threaded hole has a spherical radius of curvature.

15. A fracture fixation system according to claim 1, wherein:
said lower portion of each of said threaded holes defines a surface having a relatively high coefficient of friction.

16. A fracture fixation system according to claim 1, wherein:
said outer surface of said head of each of said pegs has a surface having a relatively high coefficient of friction.

17. A fracture fixation system according to claim 1, wherein:
said shaft of at least one of said pegs is threaded.

18. A fracture fixation plate, comprising:
a substantially rigid plate having a plurality of holes adapted to individually receive fixation pegs therein, said holes having an upper portion and a lower portion, said upper portion having a first internal thread, and said lower portion having a substantially spherical radius of curvature, said first internal threads of said holes defining respective axes at least two of said axes being oblique relative to each other.

19. A fracture fixation plate according to claim 18, wherein:
said holes includes at least threaded holes in substantially linear arrangement.

20. A fracture fixation plate according to claim 18, wherein:
said at least two axes are divergent from each other in at least one dimension.

21. A fracture fixation plate according to claim 18, wherein:
said at least two axes are divergent from each other in at least two dimensions.

22. A fracture fixation plate according to claim 18, wherein:
said at least two axes are oblique relative to each other in at least two dimensions.

23. A fracture fixation plate according to claim 22, wherein:
said at least two axes are relatively angled in rotation and inclination.

24. A fracture fixation plate according to claim 18, wherein:
each of said axes is obliquely angled relative to the other axes.

25. A fracture fixation plate according to claim 24, wherein:
each of axes is angled relative to the other in rotation and inclination.

26. A fracture fixation plate according to claim 18, wherein:
said plate including a body portion and head portion angled relative to said body portion, said body portion including at least one screw hole.

27. A fracture fixation plate according to claim 18, wherein:
said plate including a body portion and head portion angled relative to said body portion, said body portion including at least one screw hole.

28. A fracture fixation plate according to claim 18, wherein:
said plate is a sized and shaped for placement at the distal volar radius.

29. A fracture fixation plate according to claim 18, wherein:
said holes includes at least three holes in substantially linear arrangement.
